(19) United States
(12) Patent Application Publication
(10) Pub. No.: US 2006/0159634 A1
(43) Pub. Date: Jul. 20, 2006

(54) Oxazoline mixture

(75) Inventor: Franz-Leo Heinrichs, Sulzbach (DE)

Correspondence Address: CLARIANT CORPORATION;INTELLECTUAL PROPERTY DEPARTMENT, 4000 MONROE ROAD, CHARLOTTE, NC, 28205, US

(73) Assignee: Clariant GmbH

(21) Appl. No.: 11/303,627

(22) Filed: Dec. 16, 2005

(30) Foreign Application Priority Data
Dec. 18, 2004 (DE) 10 2004 061 037.1

(51) Int. Cl. A61K 8/49; C08K 5/34

(52) U.S. Cl. 424/59; 524/90; 523/161

(57) ABSTRACT

The invention relates to an oxazoline mixture comprising oxazolines of the formulae I to VI 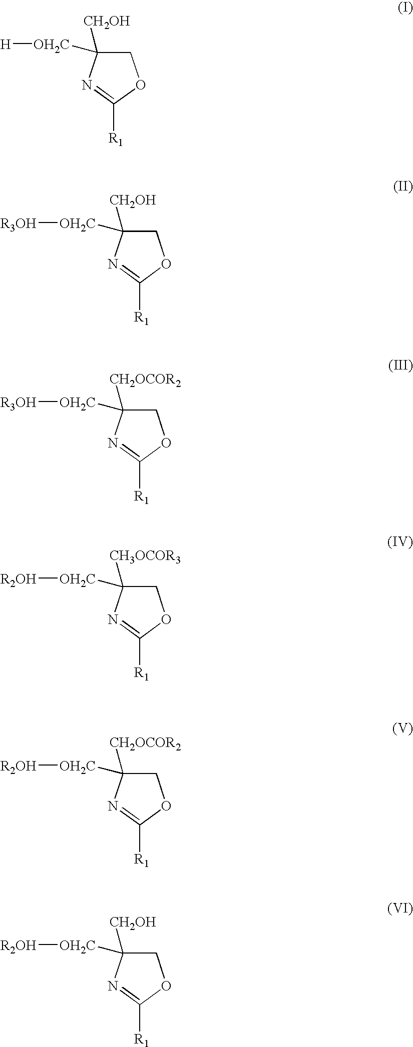
the amounts being I=from 0 to 10% mol, II=from 1 to 90% mol, III=from 1 to 98% mol, IV=from 1 to 98% mol, V=from 0 to 50% mol, VI=from 0 to 50% mol, where R₁ is the alkyl radical of a fatty acid or of a hydroxy fatty acid, R₂ is the alkyl radical of a fatty acid, hydroxy fatty acid, and/or montan wax acid, and R₃ is the alkyl radical of montan wax acid, to a process for its preparation and to the use of the mixture.

The present invention is described in the German priority application No. 10 2004 061 037.1 filed Dec. 18 , 2004, which is hereby incorporated by reference as is fully disclosed herein.

The invention relates to an oxazoline mixture, to a process for its preparation, and to use of the mixture.

Compounds of the formula VII 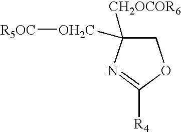
are known where R₄, R₅, and R₆ are identical, each being an alkyl radical, derived from propionic acid, caprylic acid, caproic acid, pelargonic acid, lauric acid, myristic acid, palmitic acid, oleic acid, stearic acid, hydrogenated fish oil acid, or dimer fatty acid. These products are described for use in pastes, in floorcovering cleaners, as additives in formulations for metal processing, cosmetics, paper processing, and textile processing (Römpps Chemie Lexikon [Römpp's Chemical Encyclopedia], 8th edition 1985, volume M-Pk, p. 2944, and Angus Chemie, TDS 10F Technical Datasheet (http://www.dow.com.angus)).

These compounds are prepared according to the prior art in a simple catalyst-free process via reaction of trishydroxymethylaminomethane with fatty acids, as shown in diagram I (Angus Chemie, TDS 10F Technical Datasheet (http://www.dow.com.angus)).

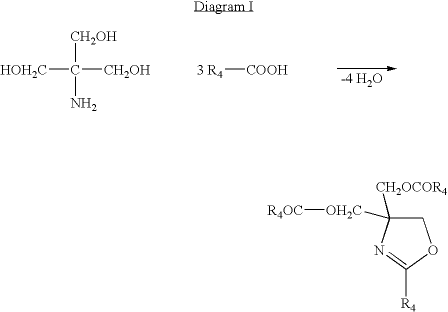

The proportion of fatty acid R₄—COOH can be selected in such a way that not all of the free hydroxy groups are reacted.

The products feature interesting properties, for example having good compatibility with many organic solvents, exhibiting basic character, and being reactive not only at the nitrogen atom but also in the oxazoline alkyl chain.

However, for applications in plastics these compounds have proven to have excessively polar character, due to the high density of functional groups. In non-polar plastics and paint systems their activity is excessively external, meaning that they are insufficiently compatible and increase their concentration at the phase boundary, where their level of activity is excessive. In polar plastics or paints their activity is excessively intemal, meaning that they have very good compatibility and have too little activity at the phase boundary. The term “external” here designates an increase in concentration at the phase boundary/surface, while the term “internal activity” here designates activity in the matrix or interaction between the polymer molecules in the matrix. The abovementioned phase boundary here designates the transition from the melt/solid matrix to air or to the surface of the tooling used in the process.

Another tendency of these compounds, by virtue of their low molecular weights, is to migrate on exposure to heat. This represents a further considerable restriction on the usefulness of these substances.

An object set was therefore to prepare products with the characteristic properties of the known oxazoline derivatives of the above formula, namely good compatibility with the organic matrix, and adequate chemical reactivity and basicity, but without their disadvantages described above, for example excessive volatility and excessive migration.

The invention therefore provides an oxazoline mixture which comprises oxazolines of the formulae 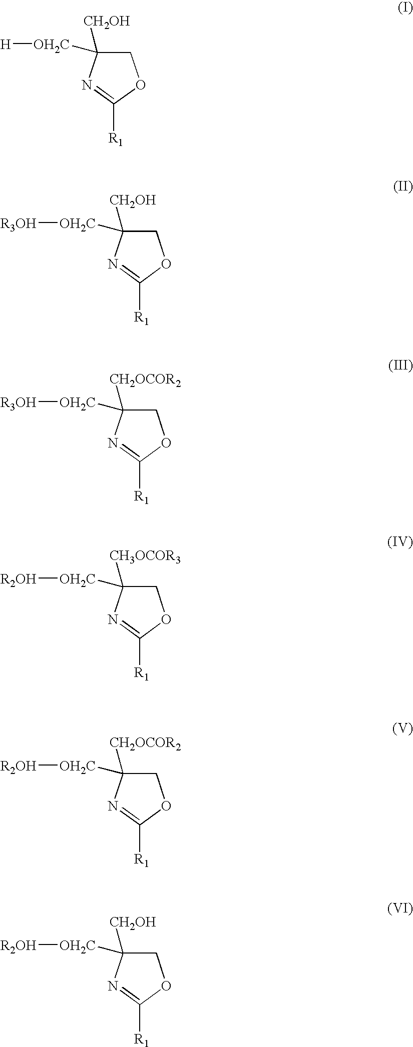
the amounts being
I=from 0 to 10% mol
11=from 1 to 90% mol
III=from 1 to 98% mol
IV=from 1 to 98% mol
V=from 0 to 50% mol
VI=from 0 to 50% mol,
   where
R₁ is the alkyl radical of a fatty acid or of a hydroxy fatty acid,
R₂ is the alkyl radical of a fatty acid, of a hydroxy fatty acid, and/or of montan wax acid, and
R₃ is the alkyl radical of montan wax acid.

The inventive oxazoline mixture preferably comprises the following amounts of the oxazolines
I=from 0 to 1% mol
II=from 2 to 50% mol
III=from 10 to 98% mol
IV=from 10 to 98% mol
V=from 0 to 20% mol
VI=from 0 to 10% mol.

It is preferable that the alkyl radicals R₁ and R₂ of the fatty acids or of the hydroxy fatty acids have from 12 to 22 carbon atoms.

It is preferable that the alkyl radicals R₁ and R₂ of the fatty acids or of the hydroxy fatty acids have linear or branched alkyl chains.

It is preferable that the alkyl radicals R₁ and R₂ of the fatty acids or of the hydroxy fatty acids are saturated or unsaturated radicals.

It is preferable that the alkyl radical of the hydroxy fatty acid is the alkyl radical of 12-hydroxystearic acid.

The invention also provides a process for preparation of an oxazoline mixture of the formulae I to VI, 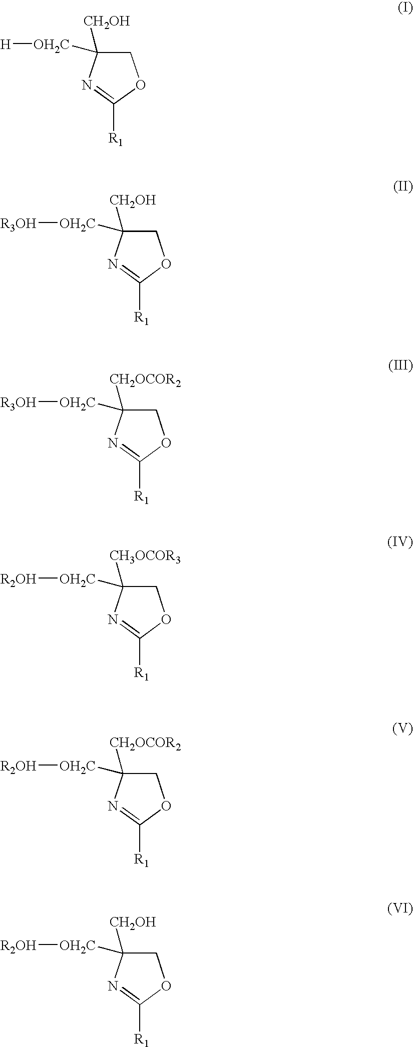
where R₁ is the alkyl radical of a fatty acid or of a hydroxy fatty acid, R₂ is the alkyl radical of a fatty acid, of a hydroxy fatty acid, and/or of montan wax acid, and
R₃ is the alkyl radical of montan wax acid, which comprises reacting trishydroxymethylaminomethane with a fatty acid and/or with a hydroxy fatty acid to give dihydroxymethyloxazolines, and then esterifying the remaining free OH groups of the dihydroxymethyloxazolines with montan wax acid in the presence of an esterification catalyst.

The reaction here proceeds in three steps in accordance with the process diagram below: 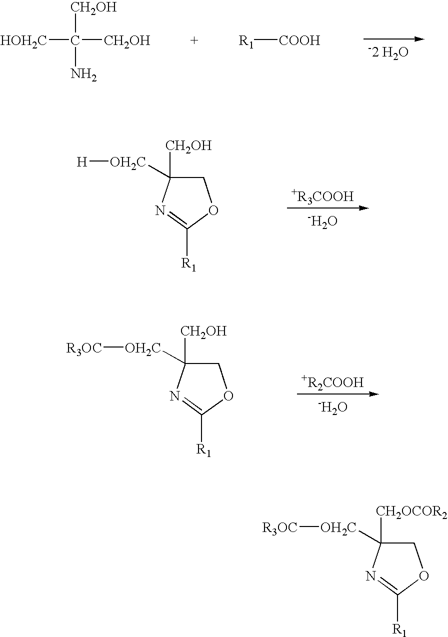
Step 1:

Reaction of trishydroxymethylaminomethane with a fatty acid and/or with a hydroxy fatty acid to give dihydroxymethyloxazolines (another possibility in this reaction being that a portion of the free OH groups are reacted).

Step 2:

Esterification with montan wax acid R₃—COOH to give the monoester (which usually contains at least 1 mol of montanic acid).

Step 3

Esterification with a carboxylic acid mixture R₂—COOH (the amount in the reaction being from 0 to 1 mol if R₂ is the alkyl radical of a fatty acid and/or of a hydroxy fatty acid; the amount in the reaction being from 1 to 2 mol if R₂ is the alkyl radical of montan wax acid).

If the free OH function has not been, or has not entirely been, reacted in step 3, the product is a mixture composed of oxazoline, monoester, and diester. The ratio of the two esters in the product mixture can be from 1:0 (pure monoester) to 0:1 (pure diester), and is preferably about 0.3:about 0.7 (30% pure monoester and 70% pure diester).

If operations are not carried out in three steps but only in two steps with a carboxylic acid mixture composed of R₃—COOH (montan wax acid) and R₂—COOH (fatty acid), the product is the monoesters composed of fatty acid and also of montan wax acid and the diesters composed of fatty acid, fatty acid/montan wax acid, and montan wax acid. The distribution of these esters in the product then depends on the constitution of the carboxylic acid mixture. The proportion of montan wax acid to be used should therefore preferably be from 1 to 2 mol, and the proportion of fatty acid to be used from 0 to 1. If monoester is intended to remain in the product mix, the proportion of the fatty acid is reduced correspondingly. The possible products are indicated below:

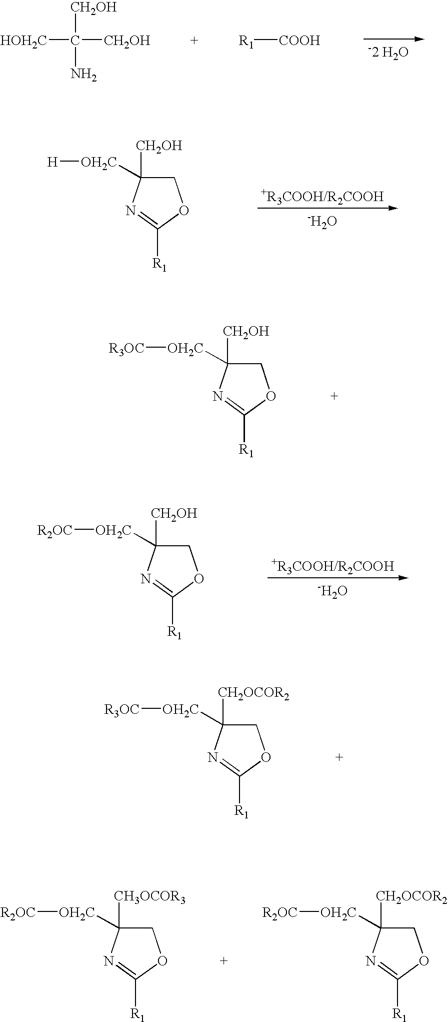

It is preferable that in the inventive process the oxazoline mixture comprises the following amounts of oxazolines of the formulae I to VI
I=from 0 to 10% mol
II=from 1 to 90% mol
III=from 1 to 98% mol
IV=from 1 to 98% mol
V=from 0 to 50% mol
VI=from 0 to 50% mol.

It is particularly preferable that in the inventive process the oxazoline mixture comprises the following amounts of oxazolines of the formulae I to VI
I=from 0 to 1% mol
II=from 2 to 50% mol
III=from 10 to 98% mol
IV=from 10 to 98% mol
V=from 0 to 20% mol
VI=from 0 to 10% mol.

It is preferable that the esterification catalyst has been selected from the group of the tin compounds, the group of the titanic esters, of zinc oxides, and/or of zinc soaps.

It is preferable that the fatty acids or hydroxy fatty acids have from 12 to 22 carbon atoms. It is preferable that the fatty acids or hydroxy fatty acids have linear or branched alkyl chains.

It is preferable that the fatty acids or hydroxy fatty acids are unsaturated acids.

It is preferable that in the inventive process 1 mol of trishydroxymethylaminomethane is first reacted with from 1 to 2.0 mol of a fatty acid and/or hydroxy fatty acid, and then the resultant product is reacted with from 1.0 to 1.9 mol of montan wax acid.

It is particularly preferable here that 1 mol of trishydroxymethylaminomethane is first reacted with from 1.1 to 1.5 mol of a fatty acid and/or hydroxy fatty acid, and then the resultant product is reacted with from 1.0 to 1.9 mol of montan wax acid.

The invention also provides the use of the oxazoline mixture as claimed in any of claims 1 to 6 as a processing aid for plastics.

The invention likewise provides the use of the oxazoline mixture as claimed in any of claims 1 to 6 as a dispersing agent for pigments and plastics additives.

It is preferable that the plastics are HI (high-impact) polystyrene, polyphenylene ether, polyamides, polyesters, polycarbonates, polyoxymethylene (POM), polyurethanes, and blends or polyblends of the type represented by ABS (acrylonitrile-butadiene-styrene) or PC/ABS (polycarbonate/acrylonitrile-butadiene-styrene) or PPE/HIPS (polyphenylene ether/HI polystyrene) plastics.

The invention also provides the use of the oxazoline mixture as claimed in any of claims 1 to 6 for production of coated organic or inorganic particles.

The invention likewise provides the use of the oxazoline mixture as claimed in any of claims 1 to 6 for production of the coated organic or inorganic particles used in plastics, in inks, and in paints.

The invention also provides organic or inorganic particles coated with the oxazoline mixture as claimed in any of claims 1 to 6.

It is preferable that the organic particles are insoluble agents such as light stabilizers, UV filters, process stabilizers, organic pigments and dyes, fibers, antioxidants, antistatic agents, and organic flame retardants.

Among the suitable organic particles are also HALS (hindered amino light stabilizers), phenols, phosphites, benzotriazoles, and aluminum phospholanes.

Organic pigments to be coated can be monoazo pigments, disazo pigments, laked azo pigments, β-naphthol pigments, naphthol AS pigments, benzimidazolone pigments, disazo condensation pigments, azo metal complex pigments, and polycyclic pigments, e.g. phthalocyanine pigments, quinacridone pigments, perylene pigments, perinone pigments, thioindigo pigments, anthanthrone pigments, anthraquinone pigments, flavanthrone pigments, indanthrone pigments, isoviolanthrone pigments, pyranthrone pigments, dioxazine pigments, quinophthalone pigments, isoindolinone pigments, isoindoline pigments, and diketopyrrolopyrrole pigments, or carbon blacks.

Organic dyes to be coated can be acid dyes, direct dyes, sulfur dyes and their leuco form, metal complex dyes, or reactive dyes, and in the case of the reactive dyes here it is also possible to use dyes reacted with nucleophiles.

It is preferable that the inorganic particles are insoluble agents such as fillers, fibers, inorganic pigments, and inorganic flame retardants.

Among the suitable inorganic particles are also lime, talc, glass fiber, iron oxide, and ammonium polyphosphates.

Examples of inorganic pigments to be coated are titanium dioxides, zinc sulfides, iron oxides, chromium oxides, ultramarine, nickel/chromium antimony titanium oxides, cobalt oxides, and also bismuth vanadates.

It is particularly preferable that the inorganic particles are titanium dioxide.

Finally, the invention also provides the use of the titanium-dioxide-containing particles coated with an oxazoline mixture as claimed in any of claims 1 to 6 for reduction of formaldehyde emission of polyoxymethylene molding compositions.

In principle, alkanolamines of the formula 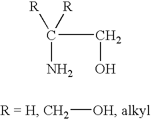
can also be used for carrying out the claimed process.

It is then preferable here that 1 mol of trishydroxymethylaminomethane is reacted with from 1 to 2 mol, in particular from 1.1 to 1.5 mol, of a fatty acid, and then that the product is reacted with 1.0 to 1.9 mol, in particular from 1.5 to 1.9 mol of montan wax acid.

Surprisingly, it has been found that the inventive products can prepared when, in a specifically modified process, in a two-stage procedure with fatty acids, the dihydroxymethyloxazolines stage is prepared and, without work-up of this precursor, the free OH groups are esterified with montan wax acid in the presence of a catalyst. These products retain a pale color, are chemically reactive at the nitrogen atom and in the oxazoline alkyl chain, exhibit basic reaction, have good compatibility with polar and non-polar plastics, but are substantially less volatile than pure fatty acid products of the prior art, and are non-migrating.

In the prior art, the substances of formula VII are prepared via reaction of trishydroxymethylaminomethane with the selected carboxylic acids at from 220 to 250° C. without catalyst. If low-molecular-weight carboxylic acids are used, the reaction has to be carried out in a pressure reactor.

The high temperatures are necessary in order to achieve sufficient reaction. In the case of the long-chain carboxylic acids having from 14 to 22 carbon atoms, operations are carried out at reduced pressure. Despite use of an inert atmosphere here, the result is color darkening.

### EXAMPLE 1 (COMPARISON)

| Raw materials | Amount (g) | mol |
|---|---|---|
| Stearic acid techn. | 810 | 3 |
| Trishydroxymethylaminomethane | 121 | 1 |

### EXAMPLE 2 (COMPARISON)

| Raw materials | Amount (g) | mol |
|---|---|---|
| Tall oil fatty acid | 840 | 3 |
| Trishydroxymethylaminomethane | 121 | 1 |

Properties of comparative products:

| | Drop | | | | |
|---|---|---|---|---|---|
| | point | Acid no. | Alkaline no. | | Volatility at |
| Properties | ° C. | mg KOH | mg KOH | Color | 300° C. |
| Example 1 | 65 | 12 | 1 | yellowish | 18.0% |
| (comparison) | | | | | |
| Example 2 | liquid | 12 | 1 | brownish | 23.0% |
| (comparison) | | | | | |

All of the constituents are charged to the reactor and then heated. Nitrogen flushing continues until 165° C. has been reached. In the range from 140-165° C., foaming has to be inhibited for safety reasons. Until the reaction temperature of 230° C. has been reached, operations are carried out at a positive pressure. The water of reaction is then discharged until the desired acid number has been reached.

### EXAMPLES OF INVENTIVE PROCESS (EXAMPLES 3 TO 6)

The respective fatty acid or hydroxy fatty acid and the trishydroxymethylamino-methane are charged to the reactor, vacuum is applied and nitrogen is supplied, and then the mixture is heated and the fatty acid is melted. Very small amounts of dispersing agent are metered into the melt, and the mixture is heated to from 40 to 80° C. above the melting point of the fatty acid. The mixture is kept at this temperature until 2 mol of water per mole of amine used have been produced.

The liquid montan wax acid is then metered into the melt, and then esterification catalyst is added and the mixture is stirred until the acid number has fallen to less than <15. The mixture is then cooled and granulated.

### EXAMPLE 3

| Raw materials | Amount (g) | mol |
|---|---|---|
| Trishydroxymethylaminomethane | 121 | 1 |
| Stearic acid techn. | 300 | 1.1 |
| Montan wax acid techn. | 750 | 1.6 |
| Esterification catalyst | 1.2 | |

### EXAMPLE 4

| Raw materials | Amount (g) | mol |
|---|---|---|
| Trishydroxymethylaminomethane | 121 | 1 |
| 12-Hydroxystearic acid techn. | 330 | 1.1 |
| Montan wax acid techn. | 750 | 1.6 |
| Esterification catalyst | 1.2 | |
| Dispersing agent | 1.2 | |

### EXAMPLE 5

| Raw materials | Amount (g) | mol |
|---|---|---|
| Trishydroxymethylaminomethane | 121 | 1 |
| 12-Hydroxystearic acid techn. | 600 | 2 |
| Montan wax acid techn. | 375 | 0.8 |
| Esterification catalyst | 1.2 | |

### EXAMPLE 6

| Raw materials | Amount (g) | mol |
|---|---|---|
| Trishydroxymethylaminomethane | 121 | 1 |
| 12-Hydroxystearic acid techn. | 900 | 3 |
| Esterification catalyst | 1.2 | |

Results

| | Drop | | | | |
|---|---|---|---|---|---|
| | point | Acid no. | Alkaline no. | | Volatility at |
| Properties | ° C. | mg KOH | mg KOH | Color | 300° C. |
| Example 3 | 74 | 15 | 0 | yellowish | 6.0% |
| Example 4 | 72 | 13 | 0 | yellowish | 5.5% |
| Example 5 | 70 | 12 | 0 | pale yellow | 9.0% |
| Example 6 | 65 | 12 | 0 | white | 15.0% |

Test methods:
Drop point: to DIN 51801/2
Acid no: to DIN 53402
Alkaline no: to DGF-M IV-4 (63)

Volatility: internal method by way of thermogravimetric analysis (TGA):

| | |
|---|---|
| Temperature range: | from 30 to 300° C. |
| Heating rate: | to 2 K/min |
| Isothermal: | 120 min at 300° C. |
| Nitrogen flushing | 50 ml/min |
| Initial weight: | 50 mg +/− 0.3 mg |
| Aluminum oxide crucible: | 150 μl |

®Licowax C is a commercially available product from Clariant GmbH based on ethylenediamine and tallow fatty acid.

### Application Example 1

Industrial polyurethane (TPU) is provided with 0.2% of lubricant and processed in the standard manner. Lubricant activity and tendency toward migration on heat-ageing were tested. The properties were evaluated by the academic-grade method, meaning that a lower-number grade indicates better lubricant action.

Processing of TPU with ®Licowax C and Product of Example 5

| | | Oxazoline of |
|---|---|---|
| | ®Licowax C | example 5 |
| Lubricant activity | 2 | 2 |
| Migration resistance at 40° C. | 4 | 2 |
| Migration resistance at 60° C. | 4-5 | 2 |

Inventive reaction products composed of trishydroxymethylaminomethane and of a fatty acid and montan wax acid exhibit good compatibility with plastics, very little tendency toward migration, and low volatility. This is improved performance when comparison is made with products of the prior art. This is apparent inter alia in a balanced distribution of internal and external activity, i.e. lubricant activity externally and dispersing activity and chemical activity intemally.

### Application Eexample 2

R 104 titanium dioxide (DuPont) was coated with the inventive product of example 4 in a heated mixer at 90° C. The proportion of the product was 2 and 4% by weight. The pigments thus modified were incorporated into POM and the product was tested for formaldehyde release. Comparison is made with standard product and with a commercially available fat-based lubricant.

Formaldehyde Emission to VDA 275

Plaques of thickness 1 mm were manufactured from the POM molding composition.

After storage for 24 h, formaldehyde emission from the plaques was determined to VDA 275 (VDA Recommendation No. 275, Dokumentation Kraftfahrwesen e.V. July 1994).

Test specimen production: the granulated polyacetal was injection-molded to give plaques of dimensions 80*50*1 mm. A Krauss Maffei KM 120/340B injection-molding machine was used with the following injection-molding parameters:
melt temperature 195° C., flow front velocity 200 mm/s, mold wall temperature 85° C., hold pressure 900 bar, hold pressure time 30 s, cooling time 10 s, back pressure from 0 to 10 bar.

Prior to testing, the test specimens were stored for 24 h in a controlled-climate cabinet at 23° C. and 50% relative humidity.

Testing: two test specimens were suspended on a stainless steel hook over 50 ml of deionized water in a 1 I flask, and kept at 60° C. for 3 h in a drying cabinet with air circulation. The test specimens were removed from the test flask. 5 ml of test specimen solution were pipetted into a test tube and the test tube was heat-conditioned at 95° C. for 10 minutes. 3 ml of acetylacetone and 3 ml of 20% strength ammonium acetate solution were then added to the test tube. Formaldehyde formed the diacetyldihydrolutidine complex with the reagents and its absorption at 412 nm was determined photometrically. Formaldehyde concentration in the test specimen solution was calculated from the absorption.

Processing of Pigmented POM (Polyoxymethylene)

| | | Amount in | VDA 275 |
|---|---|---|---|
| Polymer | Lubricant | mol % | ppm/h |
| Copolymer 2 | ®Licowax C | 0.20 | 118 |
| Copolymer 2 | Pigment + 4% product of | 0.5 | 32 |
| | Example 4 | | |
| Copolymer 2 | Pigment + 2% product of | 0.2 | 37 |
| | Example 4 | | |

Copolymer 2: ®Hostaform 27021 from Ticona GmbH ®Licowax C: ethylenebisstearamide, commercially available product from Clariant GmbH.

Use of pigment modified by the inventive product gives a markedly lower level of formaldehyde emissions when comparison is made with use of unmodified pigment in combination with standard fat-based lubricant (®Licowax C).

1. An oxazoline mixture comprising oxazolines of the formulae 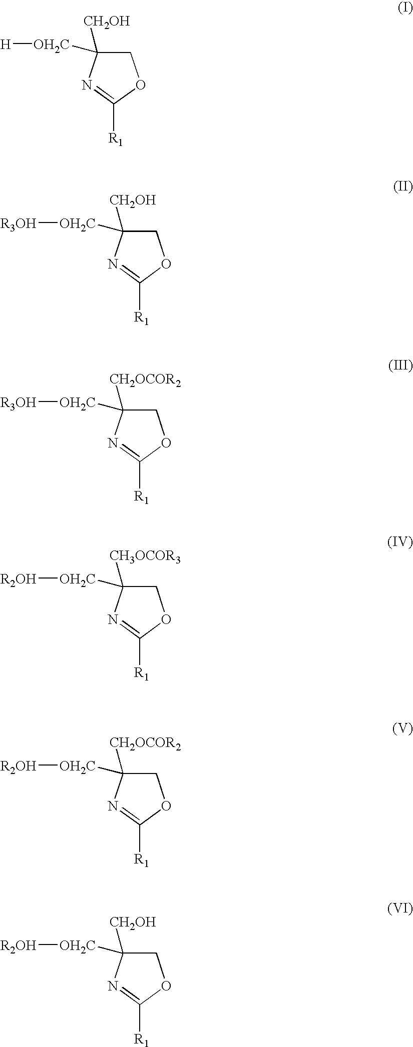
the amounts being
I=from 0 to 10% mol
II=from 1 to 90% mol
III from 1 to 98% mol
IV=from 1 to 98% mol
V=from 0 to 50% mol
VI=from 0 to 50% mol,
wherein
R₁ is the alkyl radical of a fatty acid or of a hydroxy fatty acid,
R₂ independently of one another, is the alkyl radical of a fatty acid, of a hydroxy fatty acid, or of montan wax acid, and
R₃ is the alkyl radical of montan wax acid.

2. The oxazoline mixture as claimed in claim 1, further comprising
I=from 0 to 1% mol
II=from 2 to 50% mol
III=from 10 to 98% mol
IV=from 10 to 98% mol
V=from 0 to 20% mol
VI=from 0 to 10% mol.

3. The oxazoline mixture as claimed in claim 1, wherein the alkyl radicals R₁ and R₂ of the fatty acids or of the hydroxy fatty acids have from 12 to 22 carbon atoms.

4. The oxazoline mixture as claimed in claim 1, wherein the alkyl radicals R₁ and R₂ of the fatty acids or of the hydroxy fatty acids have linear or branched alkyl chains.

5. The oxazoline mixture as claimed in claim 1, wherein the alkyl radicals R₁ and R₂ of the fatty acids or of the hydroxy fatty acids are saturated or unsaturated radicals.

6. The oxazoline mixture as claimed in claim 1, wherein the hydroxy fatty acid of at least one of R₁ and R₂ is 12-hydroxystearic acid.

7. A process for preparation of an oxazoline mixture of the formulae I to VI, 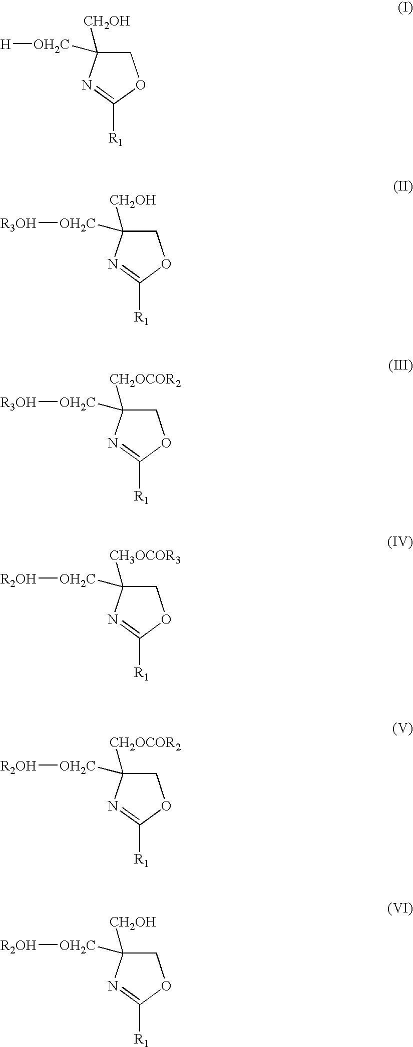
where R₁ is the alkyl radical of a fatty acid or of a hydroxy fatty acid, R₂ independently of one another is the alkyl radical of a fatty acid, of a hydroxy fatty acid, or of montan wax acid, and
R₃ is the alkyl radical of montan wax acid, comprising the steps of reacting trishydroxymethylaminomethane with at least one of a fatty acid with a hydroxy fatty acid to give dihydroxymethyloxazolines, and esterifying the remaining free OH groups of the dihydroxymethyloxazolines with montan wax acid in the presence of an esterification catalyst.

8. The process as claimed in claim 7, wherein the oxazoline mixture further comprises
I=from 0 to 10% mol
II=from 1 to 90% mol
III=from 1 to 98% mol
IV=from 1 to 98% mol
V=from 0 to 50% mol
VI=from 0 to 50% mol.

9. The process as claimed in claim 7, wherein the oxazoline mixture further comprises
I=from0to 1%mol
II=from 2 to 50% mol
III=from 10 to 98% mol
IV=from 10 to 98% mol
V=from 0 to 20% mol
VI=from 0 to 10% mol.

10. The process as claimed in claim 7, wherein the esterification catalyst is selected from the group consisting of tin compounds, titanic esters, zinc oxides, and zinc soaps.

11. The process as claimed in claim 7, wherein the fatty acids or hydroxy fatty acids have from 12 to 22 carbon atoms.

12. The process as claimed in claim 7, wherein the fatty acids or hydroxy fatty acids have linear or branched alkyl chains.

13. The process as claimed in claim 7, wherein the fatty acids or hydroxy fatty acids are saturated or unsaturated acids.

14. The process as claimed in claim 12, wherein the alkyl chains have functional groups.

15. The process as claimed in claim 7, wherein the reacting step further comprises reacting 1 mol of trishydroxymethylaminomethane with from 1 to 2.0 mol of a fatty acid, and the esterifying step further comprises reacting the dihydroxymethyloxazolines with from 1.0 to 1.9 mol of the montan wax acid.

16. The process as claimed in claim 7, wherein the reacting step further comprises reacting 1 mol of trishydroxymethylaminomethane with from 1.1 to 1.5 mol of a fatty acid, and the esterifying step further comprises reacting the dihydroxymethyloxazolines with from 1.0 to 1.9 mol of the montan wax acid.

17. A processing aid for a plastic comprising the oxazoline mixture as claimed in claim 1.

18. A dispersing agent for pigments and plastics additives comprising the oxazoline mixture as claimed in claim 1.

19. The processing aid as claimed in claim 17, wherein the plastic is selected from the group consisting of high impact polystyrene, polyphenylene ether, polyamides, polyesters, polycarbonates, polyoxymethylene, polyurethanes, and blends or polyblends of acrylonitrile-butadiene-styrene. polycarbonate/acrylonitrile-butadiene-styrene or polyphenylene ether/ high impact polystyrene plastic.

20. A process for coating organic or inorganic particles comprising the step of coating the organic or inorganic particles with the oxazoline mixture as claimed in claim 1.

21. A composition comprising the coated organic or inorganic particles as claimed in claim 24, wherein the composition is selected from the group consisting of plastics, inks, and paints.

22. An insoluble agent for a composition, wherein the insoluble agent comprises the coated organic particles as claimed in claim 24 and wherein the composition is selected from the group consisting of light stabilizers, UV filters, process stabilizers, organic pigments, fibers, antioxidants, antistatic agents, and organic flame retardants.

23. An insoluble agent for a composition, wherein the insoluble agent comprises the coated inorganic particles as claimed in claim 24 and wherein the composition is selected from the group consisting of fillers, fibers, inorganic pigments, and inorganic flame retardants.

24. Coated organic or inorganic particles coated with an oxazoline mixture as claimed in claim 1.

25. A composition comprising the coated organic particles as claimed in claim 24, wherein the composition is selected from the group consisting of light stabilizers, UV filters, process stabilizers, organic pigments, fibers, antioxidants, antistatic agents, and organic flame retardants.

26. A composition comprising the coated inorganic particles as claimed in claim 24, wherein the composition is selected from the group consisting of fillers, fibers, inorganic pigments, and inorganic flame retardants.

27. The particle as claimed in claim 26, wherein the inorganic particles are titanium dioxide.

28. A method for reducing the formaldehyde emission of a polyoxymethylene molding composition comprising the step of adding inorganic titanium dioxide containing particles coated with an oxazoline mixture as claimed in claim 1 to the polvoxymethylene molding composition during production of the polyoxymethylene molding composition.

29. The processing aid as claimed in claim 17, wherein the plastics addtive has at least one plastic selected from the group consisting of high impact polystyrene, polyphenylene ether, polyamides, polyesters, polycarbonates, polyoxymethylene, polyurethanes, and blends or polyblends of acrylonitrile-butadiene-styrene, polycarbonate/acrylonitrile-butadiene-styrene or polyphenylene ether/high impact polystyrene plastic.

30. A polyoxymethylene molding composition made in accordance with the process of claim 28.
